(19) United States
(12) Patent Application Publication
(10) Pub. No.: US 2024/0000591 A1
(43) Pub. Date: Jan. 4, 2024

(54) RESILIENCE ADAPTIVE STENT DELIVERY DEVICE AND METHODS

(71) Applicant: Merit Medical Systems, Inc., South Jordan, UT (US)

(72) Inventors: Bryan Elwood, South Jordan, UT (US); Zeke Eller, Plano, TX (US)

(21) Appl. No.: 18/342,311

(22) Filed: Jun. 27, 2023

Related U.S. Application Data
(60) Provisional application No. 63/367,296, filed on Jun. 29, 2022.

(51) Int. Cl. A61F 2/95; A61F 2/966

(52) U.S. Cl. CPC A61F 2/9517; A61F 2/966

(57) ABSTRACT

Devices and methods used to deploy a protheses, such as a stent, are disclosed. The devices include a stent deployment device including a handle assembly coupled to a delivery catheter assembly. The handle assembly includes an actuator operably coupled to a ratchet slide. A carrier coupled to an outer sheath of the delivery catheter assembly is axially displaced by the ratchet slide when the actuator is depressed. A distal deployment button allows proximal displacement of the carrier when in an actuated position to deploy a distal portion of the prosthesis. A proximal deployment button allows proximal displacement of the carrier when in an actuated position to deploy a proximal portion of the prosthesis. The proximal deployment button includes a stop member that is axially adjustable relative to the distal deployment button.

### RELATED APPLICATIONS

This application claims priority to U.S. Provisional Application No. 63/367,296, filed Jun. 29, 2022, titled RESILIENCE ADAPTIVE STENT DELIVERY DEVICE AND METHODS, which is incorporated herein by reference in its entirety.

### TECHNICAL FIELD

The present disclosure relates generally to medical devices. More specifically, the present disclosure relates to prosthesis deployment devices, including deployment devices for prostheses such as stents and stent-grafts.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a stent deployment device.

FIG. 2 is a perspective view of an embodiment of a handle assembly of the stent deployment device of FIG. 1.

FIG. 3 is a cross-sectional side view of the handle assembly of FIG. 2.

FIG. 4A is a perspective view of an embodiment of a ratchet slide of the handle assembly of FIG. 3.

FIG. 4B is a cross-sectional side view of the ratchet slide of FIG. 4A.

FIG. 5 is a perspective view of an embodiment of a carrier of the handle assembly of FIG. 3.

FIG. 6 is a side view of an embodiment of a portion of the handle assembly of FIG. 3.

FIG. 6A is a side view of a section of the portion of the handle assembly of FIG. 6.

FIG. 7 is a top cross-sectional view of the handle assembly of FIG. 2.

FIG. 8A is a bottom perspective view of an embodiment of a distal deployment button of FIG. 2.

FIG. 8B is a top perspective view of the distal deployment button of FIG. 8A.

FIG. 9A is a bottom perspective view of an embodiment of a proximal deployment button of FIG. 2.

FIG. 9B is a top perspective view of the proximal deployment button of FIG. 9A.

FIG. 10 is a perspective view of an embodiment of a pinion gear of the proximal deployment button of FIG. 9A.

FIG. 11 is a perspective view of the proximal deployment button of FIG. 9A disposed within the handle assembly.

FIG. 12A is a perspective view of the handle assembly with a portion of the housing removed and the distal deployment button in a non-actuated position.

FIG. 12B is a perspective view of the handle assembly with a portion of the housing removed and the distal deployment button in an actuated position.

FIG. 12C is a perspective view of the handle assembly with a portion of the housing removed and the proximal deployment button in a non-actuated position.

FIG. 12D is a perspective view of the handle assembly with a portion of the housing removed and the proximal deployment button in an actuated position.

FIG. 13A is a perspective view of another embodiment of the proximal deployment button in a non-actuated position.

FIG. 13B is a perspective view of the proximal deployment button of FIG. 13A in an actuated position.

FIG. 14A is a perspective view of another embodiment of the proximal deployment button in a non-actuated position.

FIG. 14B is a perspective view of the proximal deployment button of FIG. 14A in an actuated position.

### DETAILED DESCRIPTION

Deployment devices may be configured to deliver a medical appliance to a location within a patient's body and deploy the medical appliance within the patient's body. Though specific examples recited herein may refer to deployment of devices within the gastrointestinal tract (including, for example, within the esophagus, intestines, stomach, small bowel, colon, and biliary duct), analogous concepts and devices may be used in various other locations within the body, including for placement and deployment of medical appliances in the respiratory system (including, for example, within the trachea, bronchial tubes, lungs, nasal passages, and sinuses); vasculature; or any other location within the body, both within bodily lumens (for example, the ureter, the urethra, and/or any of the lumens discussed above) and within other bodily structures.

Furthermore, though specific examples herein may refer to deployment of prostheses such as stents, deployment of a wide variety of medical appliances are within the scope of this disclosure, including stents, stent-grafts, shunts, grafts, and so forth. Additionally, the deployment device disclosed herein may be configured to deliver and deploy self-expanding medical appliances, including stents configured to expand within a bodily lumen upon deployment.

As used herein, delivery of a medical appliance generally refers to placement of a medical appliance in the body, including displacement of the appliance along a bodily lumen to a treatment site. For example, delivery includes displacement of a crimped stent along a bodily lumen from an insertion site to a treatment location. Deployment of a medical appliance refers to placement of the medical appliance within the body such that the medical appliance interacts with the body at the point of treatment. For example, deployment includes releasing a crimped or otherwise constrained self-expanding stent from a deployment device such that the stent expands and contacts a bodily lumen.

Deployment devices within the scope of this disclosure may be configured to incrementally deploy a medical appliance. Incremental deployment may facilitate desired placement of the medical appliance due to the degree of control afforded a practitioner during deployment. A practitioner may, for example, desire to deploy a portion of a stent, make adjustments to placement within the bodily lumen, or confirm the location of the stent prior to deploying the remaining portion of the stent. Such processes may be iterative, with a practitioner deploying a portion of a stent, confirming placement, deploying an additional portion, again confirming placement, and so forth until the stent is fully deployed.

Deployment devices within the scope of this disclosure may be configured to provide visual, audible, tactile, or other feedback relating to the degree to which a medical appliance has been deployed. Multiple types of feedback may enhance a practitioner's level of control over the procedure due to the multiple indications regarding location or degree of deployment of the medical appliance.

Deployment devices within the scope of this disclosure may provide a degree of mechanical advantage during deployment, for example, through the use of levers to decrease the force used to deploy a device. Mechanical advantage may thus increase a user's comfort and level of control during use. Still further, deployment devices within the scope of this disclosure may be ergonomically designed, presenting an actuation input disposed such that a practitioner can directly engage and utilize the device, without repositioning his or her hand or body. Deployment devices within the scope of this disclosure may also be configured for one-handed actuation and may be configured for ambidextrous use.

Deployment devices within the scope of this disclosure may provide a degree of adaptive resilience during deployment of a stent. For example, a proximal deployment button or safety member may be selectively adjustable relative to a distal deployment button or safety member to provide a variable deployment distance of a distal portion of a stent or a first stent dependent upon a length of the stent. The deployment distance may be set during manufacture of the deployment devices. In another embodiment, the deployment distance may be set by the user.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

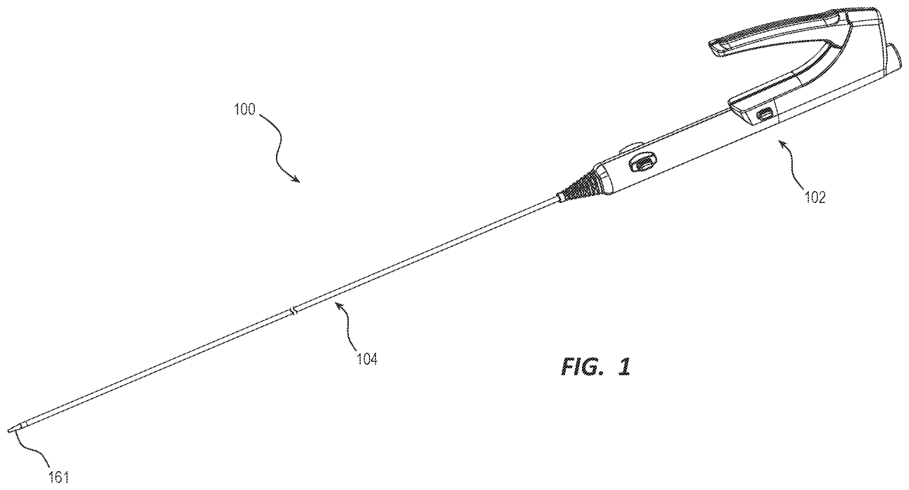

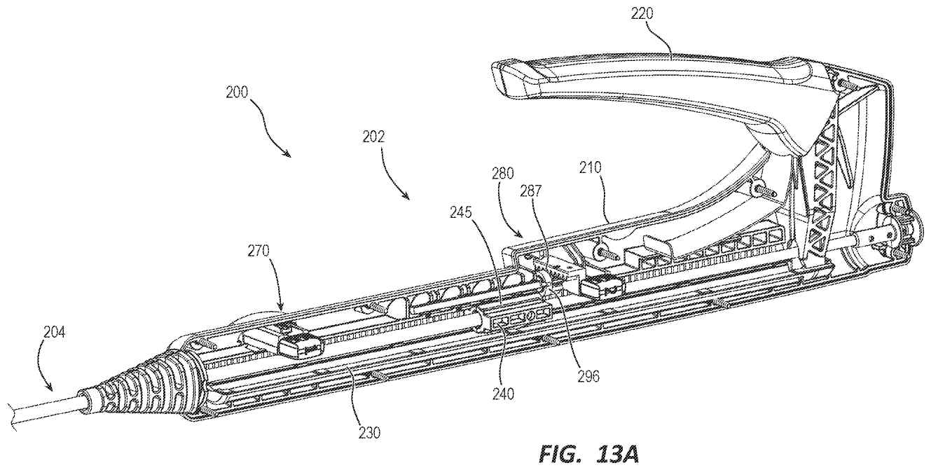

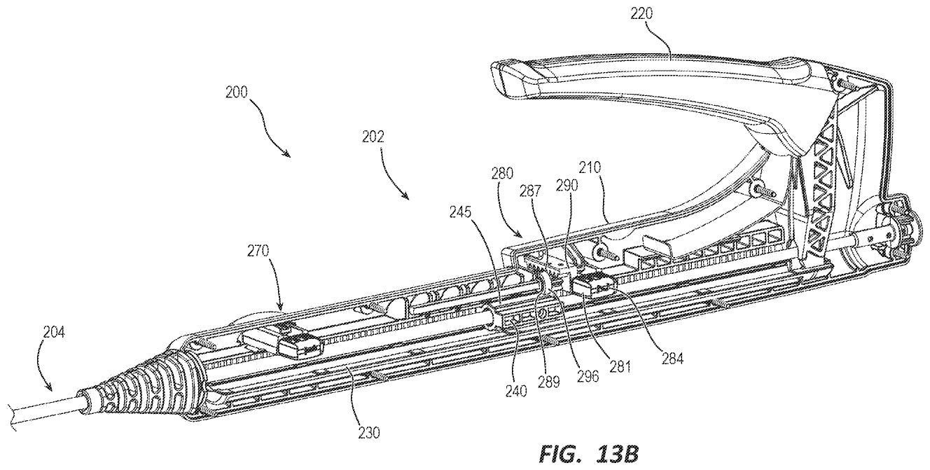

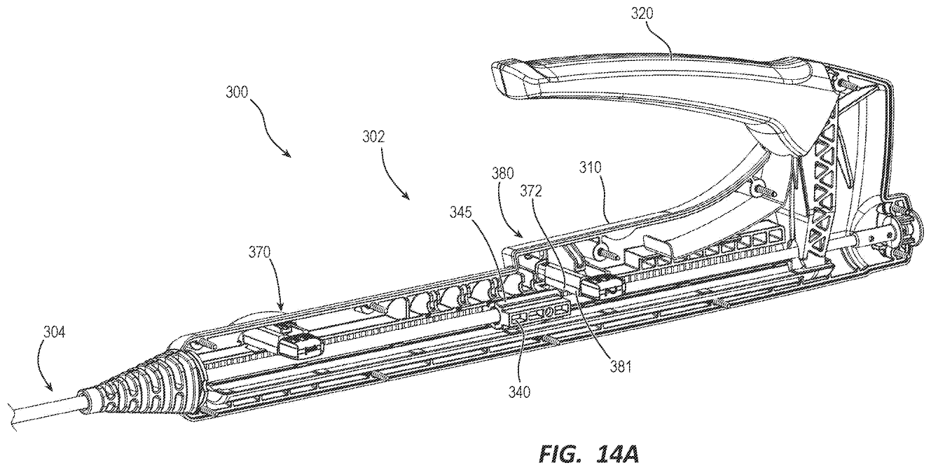

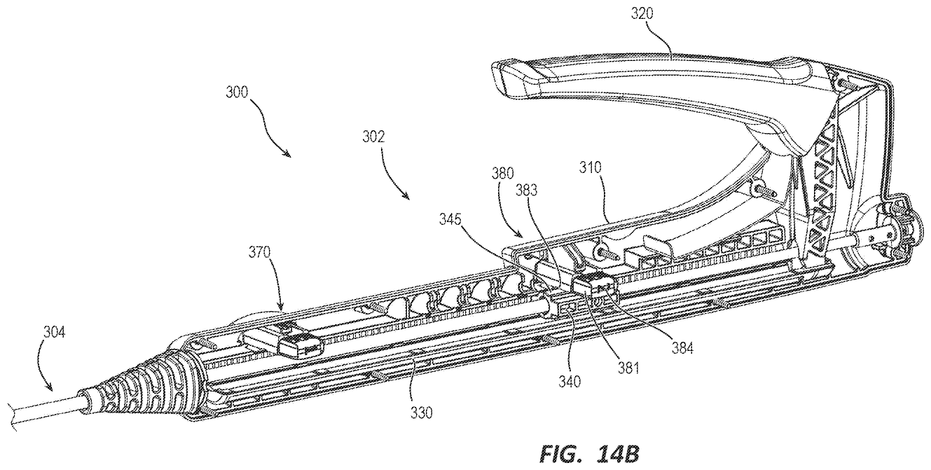

FIGS. 1-12C illustrate an embodiment of a stent deployment device including various components such as distal and proximal deployment buttons and a method of use. FIGS. 13A and 13B illustrate another embodiment of a stent deployment device having another embodiment of a proximal deployment button. FIGS. 14A and 14B illustrate another embodiment of a stent deployment device having another embodiment of a proximal deployment button. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIG. 1 is a perspective view of a stent deployment device 100. The stent deployment device 100 comprises a handle assembly 102 adjacent the proximal end of the stent deployment device 100. An elongate delivery catheter assembly 104 extends distally from the handle assembly 102 to a distal tip or delivery tip 161. The handle assembly 102 may provide a proximal user input, with one or more components configured to allow a practitioner to deploy or otherwise manipulate a prosthesis (e.g., stent) disposed within the delivery catheter assembly 104.

While in use, the handle assembly 102 of the stent deployment device 100 may be disposed outside of a patient's body, while the delivery catheter assembly 104 is advanced to a treatment location within the patient's body. As detailed below, a stent may be disposed within a portion of the delivery catheter assembly 104 such that a practitioner may deploy the stent from a distal end of the delivery catheter assembly 104 through manipulation of one or more components of the handle assembly 102.

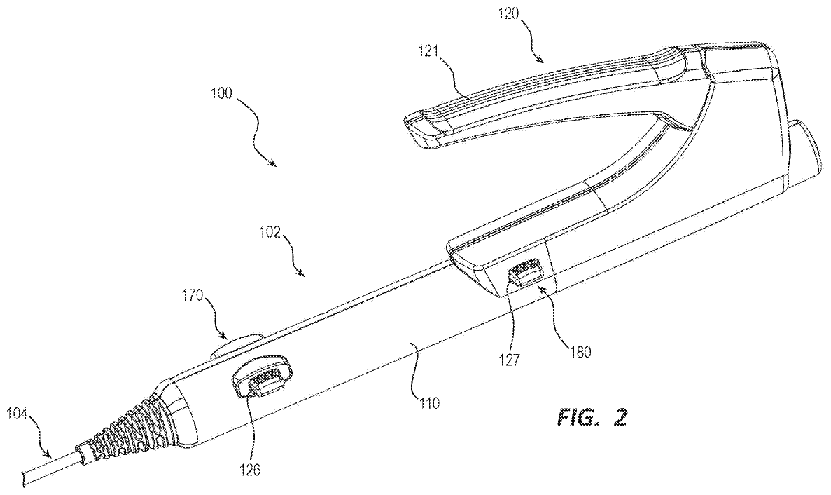

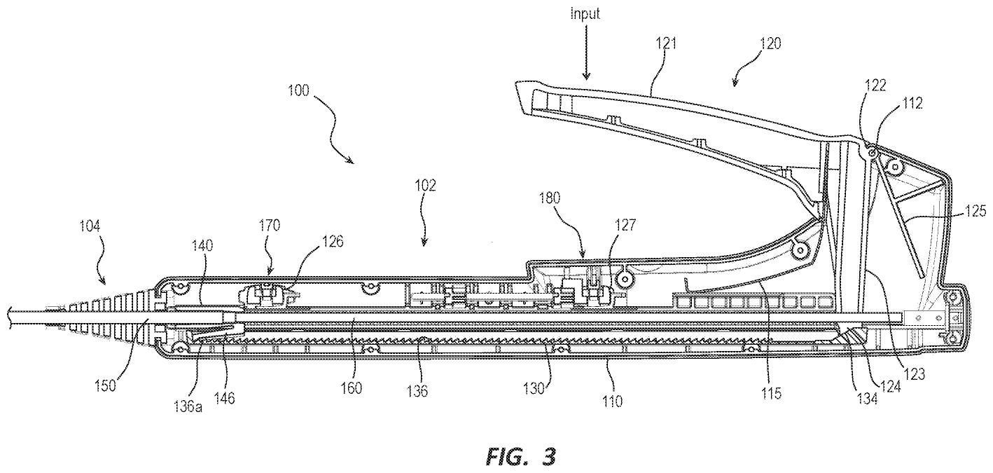

FIG. 2 is a perspective view and FIG. 3 is a cross-sectional side view of a proximal portion of the stent deployment device 100 of FIG. 1. Specifically, FIG. 3 is a side view of a portion of the stent deployment device 100 of FIG. 1, taken through a cross-sectional plane extending vertically and intersecting a longitudinal axis of the stent deployment device 100, when the stent deployment device 100 is positioned as shown in FIG. 1. The longitudinal axis of the stent deployment device 100 extends along the center of the delivery catheter assembly 104, including along the center of components of the delivery catheter assembly 104 which overlap with the handle assembly 102.

As the handle assembly 102 is configured to be grasped or otherwise manipulated by a user and the delivery catheter assembly 104 is configured to extend to a treatment location within a patient's body, along the longitudinal axis, the delivery catheter assembly 104 extends in a distal direction away from the handle assembly 102. The proximal direction is opposite, correlating to a direction defined along the longitudinal axis, extending from the distal tip 161 (shown in FIG. 1) toward the handle assembly 102.

FIG. 3 depicts various internal components of the handle assembly 102, exposed by the cross-sectional view. A portion of the delivery catheter assembly 104 is also shown extending from the handle assembly 102. The handle assembly 102 comprises a housing 110. The housing 110 surrounds certain components of the handle assembly 102, as shown in FIG. 2, providing a grip surface for a practitioner.

An actuator 120 is operably coupled to the housing 110. Manipulation of the actuator 120 with respect to the housing 110 may be configured to deploy the stent, as further detailed below. In the depicted embodiment, the actuator 120 is rotatably coupled to the housing 110 by a pin 112. The pin 112 extends from the housing 110 and may be integrally formed with one or more other portions of the housing 110. As shown, the pin 112 extends through a pin aperture 122 in the actuator 120. As discussed above in reference to the actuator 120 and the housing 110, other arrangements for operably coupling the actuator 120 and the housing 110 are also within the scope of this disclosure.

The actuator 120 comprises an input portion 121 extending from the pin aperture 122. In the depicted embodiment, the input portion 121 comprises a surface, at least partially exposed with respect to the housing 110. In operation, a user may manipulate the actuator 120 by exerting a force on the input portion 121, illustrated by the arrow labeled “input” in FIG. 3, displacing the input portion 121 generally toward the longitudinal axis of the stent deployment device 100 and causing the actuator 120 to rotate about the pin 112 with respect to the housing 110. Displacement of the actuator 120 due to a force such as illustrated by the arrow labeled “input” corresponds to “depression” of the actuator 120 or “depression of the actuator 120 with respect to the housing 110.”

The actuator 120 may further comprise a transfer arm 123 extending from the pin aperture 122. The transfer arm 123 may be rigidly coupled to the input portion 121, including embodiments wherein both the transfer arm 123 and an input portion 121 are integrally formed with the rest of the actuator 120. The transfer arm 123 extends to a ratchet slide engaging portion 124. Depression of the input portion 121, in the direction shown by the arrow labeled “input,” displaces the transfer arm 123 as the actuator 120 is rotated about the pin 112.

Depression of the input portion 121 thus causes displacement of the ratchet slide engaging portion 124 with respect to the housing 110. This displacement of the ratchet slide engaging portion 124 can be understood as rotation about the pin 112 having a proximal translation component and a vertical translation component, as rotation of the input portion 121 in the direction indicated by the arrow labeled “input” will displace (with respect to the housing 110) the ratchet slide engaging portion 124 both proximally and vertically.

A spring 115 may be disposed between the actuator 120 and the housing 110. The spring 115 may be configured to resist displacement of the actuator 120 in the direction indicated by the arrow labeled “input” and may be configured to return the actuator 120 to the relative position shown in FIGS. 2 and 3 after it has been depressed by a user. When the handle assembly 102 is unconstrained, the spring 115 may thus maintain (or return to) the relative position of the actuator 120 with respect to the housing 110 as shown in FIGS. 2 and 3.

As the actuator 120 is depressed with respect to the housing 110, the spring 115 compresses and the ratchet slide engaging portion 124 is displaced as described above. Again, the displacement of the ratchet slide engaging portion 124 with respect to the housing 110 can be understood as having a proximal component and a vertical component.

The ratchet slide engaging portion 124 may be operably coupled to a ratchet slide 130 such that displacement of the ratchet slide engaging portion 124 likewise displaces the ratchet slide 130. The ratchet slide 130 may be constrained such that the ratchet slide 130 is configured only for proximal or distal displacement with respect to the housing 110. Thus, operable coupling of the ratchet slide engaging portion 124 to the ratchet slide 130 may allow for sliding interaction between the ratchet slide engaging portion 124 and the ratchet slide 130 such that only the proximal or distal component of the displacement of the ratchet slide engaging portion 124 is transferred to the ratchet slide 130. Stated another way, the ratchet slide 130 may be displaced in a direction parallel to the longitudinal axis of the deployment device 100 while the input displacement may be at an angle to the longitudinal axis of the deployment device 100. It is noted that, in the configuration shown in FIG. 3, one or more deployment buttons or safety members 170, 180 may prevent proximal displacement of the ratchet slide 130. Discussion herein relating to displacement of the ratchet slide 130 and related components may thus be understood as disclosure relevant to a configuration of the handle assembly 102 in which the one or more of the deployment buttons 170, 180 have been actuated.

As the actuator 120 is depressed with respect to the housing 110, the ratchet slide 130 may thus be proximally displaced with respect to the housing 110. One or both of the ratchet slide 130 and actuator 120 may also interact with the housing 110 such that there is a positive stop 125 to arrest the depression of the actuator 120 and/or proximal displacement of the ratchet slide 130. This positive stop may be an engaging ledge, shoulder, lug, detent, or other feature coupled to the housing 110, including features integrally formed on the housing 110. In the depicted embodiment of FIG. 3, the positive stop 125 is a structure disposed proximal to the actuator 120 and configured to engage the transfer arm 123 to arrest depression of the actuator 120. In other embodiments, the positive stop 125 can be disposed proximally of a proximal end of the ratchet slide 130. For example, the proximal end of the ratchet slide 130 can interact with a portion of the housing 110 (a ledge, shoulder, etc.) disposed proximally of the proximal end of the ratchet slide 130. Accordingly, the handle assembly 102 may be configured such that the ratchet slide 130 is displaced or “travels” as much as possible during depression of the actuator 120.

A full stroke of the actuator 120 may thus correspond to displacement from the unconstrained position shown in FIG. 3, to the positive stop caused by interaction with the positive stop 125 when the actuator 120 is depressed. A partial stroke of the actuator 120 may correspond to displacement from the unconstrained position shown in FIG. 3, to each and/or any position prior to the positive stop 125 caused by interaction with the positive stop 125 when the actuator 120 is depressed. Release of the actuator 120 following a full stroke or a partial stroke may then result in a return of the actuator 120 to the unconstrained state, due to the biasing force provided by the spring 115. The unconstrained state shown in FIG. 3 refers to lack of constraint due to user input. In this state, the spring 115 may be partially compressed, and interaction between the actuator 120 and the housing 110 may prevent rotation of the actuator 120 about the pin 112 in the opposite direction to depression of the actuator 120, or the return direction. In other words, interaction between the actuator 120 and the housing 110 (or features of the housing 110) may create a positive stop to the return motion of the actuator 120 as well.

With continued reference to FIGS. 2 and 3, the ratchet slide 130 may thus be proximally displaced during depression of the actuator 120. Again, such displacement may correspond to a configuration in which the distal deployment button 170 has been actuated. Proximal displacement of the ratchet slide 130 may also proximally displace a carrier 140 due to interaction between one or more carrier engaging ratchet lugs 136 on the ratchet slide 130 and a ratchet slide engaging arm 146 coupled to the carrier 140. In some embodiments, the carrier 140 may be coupled to an outer sheath 150 of the delivery catheter assembly 104. For example, the carrier 140 may be fixedly and/or rigidly coupled to the outer sheath 150. In certain embodiments, an inner sheath 160 of the delivery catheter assembly 104 may be coupled to the handle assembly 102. For example, the inner sheath 160 may be fixedly and/or rigidly coupled to the handle assembly 102.

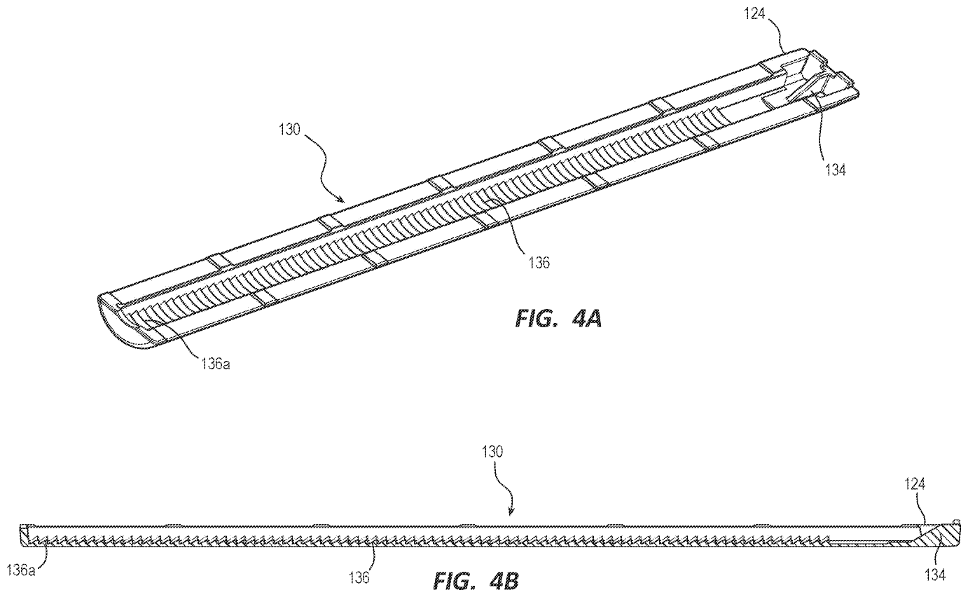

FIG. 4A is a perspective view of the ratchet slide 130 of the deployment device 100 of FIG. 3. FIG. 4B is a cross-sectional view of the ratchet slide 130 of FIG. 4A, taken through a vertical plane disposed along a longitudinal centerline of the ratchet slide 130. When the ratchet slide 130 is disposed within the handle assembly 102 of FIG. 3, this cross-sectional plane would intersect the longitudinal axis of the deployment device 100.

As shown in FIGS. 3, 4A, and 4B, the ratchet slide 130 may comprise a plurality of carrier engaging ratchet lugs 136. The carrier engaging ratchet lugs 136 may be spaced at even intervals along the longitudinal direction of the ratchet slide 130. As depicted, the plurality of carrier engaging ratchet lugs 136 may be disposed semi-continuously. For example, consecutive carrier engaging ratchet lugs 136 may be spaced about 5 mm or less from each other, about 4 mm or less from each other, about 3 mm or less from each other, about 2 mm or less from each other, about 1 mm or less from each other, or any other suitable distance from each other. In the figures, exemplary carrier engaging ratchet lugs are denoted with reference numeral 136, while the distal most carrier engaging ratchet lug, disposed at the distal end of the ratchet slide 130, is denoted with reference numeral 136a.

As noted above, interaction between the ratchet slide engaging portion 124 of the actuator 120 and the ratchet slide 130 may proximally displace the ratchet slide 130 with respect to the housing 110. Engagement between the carrier 140 and one of the carrier engaging ratchet lugs 136 may also proximally displace the carrier 140 as the ratchet slide 130 is proximally displaced with respect to the housing 110. In the configuration of FIG. 3, a ratchet slide engaging arm 146 of the carrier 140 is engaged with the distal most carrier engaging ratchet lug 136a.

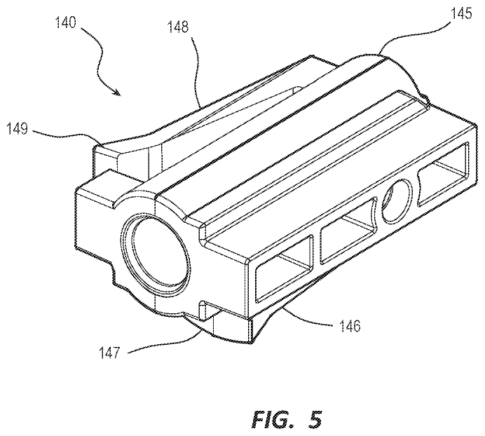

FIG. 5 is a perspective view of the carrier 140 of the deployment device 100 of FIG. 3. As shown in FIG. 5, the ratchet slide engaging arm 146 extends radially away from a longitudinal axis of the carrier 140. When the carrier 140 is disposed within the handle assembly 102 of FIG. 3, the longitudinal axis of the carrier 140 is disposed along the longitudinal axis of the deployment device 100.

As depicted, the ratchet slide engaging arm 146 comprises an angled portion or “toenail” portion 147 at a distal end of the ratchet slide engaging arm 146. As shown, the angled portion 147 extends radially away from the longitudinal axis of the carrier 140 at a greater angle than the radial extension of the ratchet slide engaging arm 146 in relation to the longitudinal axis of the carrier 140. In some embodiments, the angled portion 147 can enhance engagement between the ratchet slide engaging arm 146 and a given carrier engaging ratchet lug 136 as compared to a ratchet slide engaging arm lacking an angled portion. For example, due at least in part to the semi-continuous disposition of the plurality of the carrier engaging ratchet lugs 136 (as shown in FIGS. 4A and 4B), the angled portion 147 of the ratchet slide engaging arm 146 can allow or permit the ratchet slide engaging arm 146 to deflect radially adjacent to or against at least a portion of the ratchet slide 130 at or adjacent the given carrier engaging ratchet lug 136. The angled portion 147 can provide clearance for the ratchet slide engaging arm 146, allowing the angled portion to engage carrier engaging ratchet lugs 136 (even when closely spaced) without adjacent lugs interfering with the position of the ratchet slide engaging arm 146 and preventing full engagement.

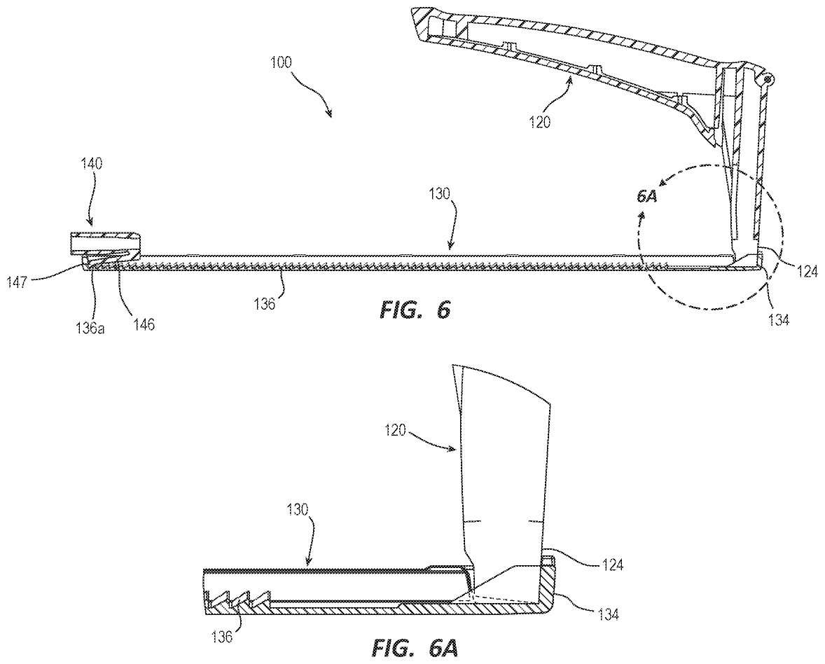

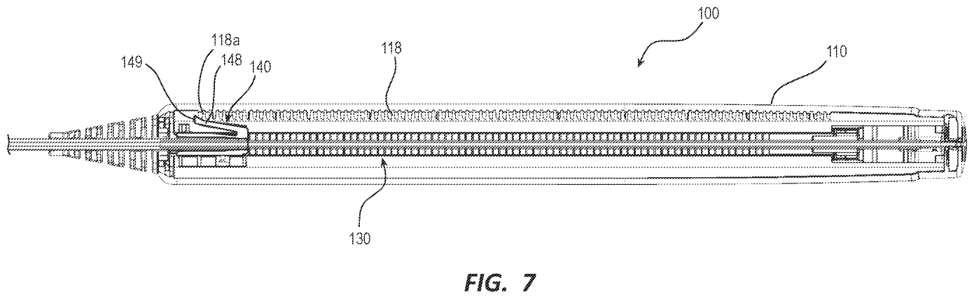

FIG. 6 is a cross-sectional view of a portion of the deployment device 100 shown in FIG. 3. Specifically, the actuator 120, the ratchet slide 130, and the carrier 140 are shown in FIG. 6, in the same relative positions, and along the same cross-sectional plane as in FIG. 3. FIG. 6A is a partial cut-away view of a portion of the cross-sectional view of FIG. 6. As shown, a portion of the ratchet slide 130 has been cut away in this view to show an engagement of the ratchet slide engaging portion 124 with an actuator engaging opening 134.

Referring to FIGS. 2-6A, during depression of the actuator 120 with respect to the housing 110, the actuator 120 rotates around the pin aperture 122. This rotation causes displacement of the ratchet slide engaging portion 124 of the actuator 120. The component of this displacement correlating to proximal displacement of the ratchet slide engaging portion 124 also proximally translates the ratchet slide 130 due to interaction between the ratchet slide engaging portion 124 of the actuator 120 and the actuator engaging opening 134 of the ratchet slide 130. Stated another way, the walls or faces that define the actuator engaging opening 134 may contact the ratchet slide engaging portion 124 such that the ratchet slide 130 is displaced when the actuator 120 is displaced.

Proximal displacement of the ratchet slide 130 also proximally displaces the carrier 140 due to interaction between the carrier engaging ratchet lugs 136 and the ratchet slide engaging arm 146. In the depicted embodiment, a distal surface of the angled portion 147 of the ratchet slide engaging arm 146 is in contact with a proximal face of the distal most carrier engaging ratchet lug 136a. This contact exerts proximal force on the distal surface of the angled portion 147 of the ratchet slide engaging arm 146, displacing the carrier 140 in a proximal direction. Accordingly, the ratchet slide 130 and carrier 140 will move proximally until the actuator 120 reaches the end of the stroke (e.g., either a partial stroke or a full stroke).

FIG. 7 is a cross-sectional view of the housing 110 and the carrier 140 in the same relative positions shown in FIG. 3. The cross-sectional plane of FIG. 7 extends along the longitudinal axis of the deployment device 100; however, the cross-sectional plane of FIG. 7 extends horizontally, orthogonal to the cross-sectional planes of FIGS. 3, 4B, and 6.

As shown in FIG. 7, the carrier 140 comprises a housing engaging arm 148 extending radially away from a longitudinal axis of the carrier 140. The housing 110 comprises a plurality of carrier engaging housing lugs 118. In FIG. 7, exemplary carrier engaging housing lugs are denoted by reference numeral 118, with the distal most carrier engaging housing lug denoted by reference numeral 118a.

As depicted in FIGS. 5 and 7, the housing engaging arm 148 comprises an angled portion or “toenail” portion 149 at a distal end of the housing engaging arm 148. As shown, the angled portion 149 extends radially away from the longitudinal axis of the carrier 140 at a greater angle than the radial extension of the housing engaging arm 148 in relation to the longitudinal axis of the carrier 140. In some embodiments, the angled portion 149 can enhance engagement between the housing engaging arm 148 and a given carrier engaging housing lug 118 as compared to a housing engaging arm lacking an angled portion. For example, due at least in part to the semi-continuous disposition of the plurality of the carrier engaging housing lugs 118, the angled portion 149 of the housing engaging arm 148 can allow or permit the housing engaging arm 148 to deflect radially adjacent to or against at least a portion of the ratchet slide 130 at or adjacent the given carrier engaging housing lug 118. As with the angled portion 147 discussed above, the angled portion 149 can provide clearance for the housing engaging arm 148, allowing the angled portion 149 to engage carrier engaging housing lugs 118 (even when closely spaced) without adjacent lugs interfering with the position of the housing engaging arm 148 and preventing full engagement.

Referring to FIGS. 3-7, as interaction between the actuator 120, ratchet slide 130, and carrier 140 displaces the carrier 140 with respect to the housing 110 (as shown and described above), the housing engaging arm 148 (shown in FIG. 5) of the carrier 140 will deflect radially inward due to contact with one of the carrier engaging housing lugs 118. For example, from the position shown in FIG. 7, as interaction between the distal most carrier engaging ratchet lug 136a and the ratchet slide engaging arm 146 of the carrier 140 draws the carrier 140 proximally, the distal most carrier engaging housing lug 118a causes the housing engaging arm 148 to displace radially inward. The housing engaging arm 148 will continue to deflect radially inward until the distal end of the housing engaging arm 148 is positioned proximal of the distal most carrier engaging housing lug 118a, at which point the housing engaging arm 148 will return to the radially outward configuration shown in FIG. 7. The point at which the housing engaging arm 148 moves proximally of the distal most carrier engaging housing lug 118a may correspond to the stroke of the actuator 120 (e.g., a partial stroke or a full stroke), such that engagement between the housing engaging arm 148 and the next carrier engaging housing lug 118 (moving in a proximal direction) occurs at the end of the stroke. In some embodiments, each carrier engaging housing lug 118 (or at least a portion of each of the carrier engaging housing lugs 118) may be disposed such that a position of the carrier engaging housing lug 118 corresponds to a position of a carrier engaging ratchet lug 136.

Further, a stroke of the actuator 120 can correspond to displacement of the carrier 140 past multiple carrier engaging housing lugs 118. For closely spaced carrier engaging housing lugs 118, the actuator 120 may thus be configured to displace the carrier 140 over a semi-continuous range as the carrier 140 is advanced along the carrier engaging housing lugs 118. Partially depressing the actuator 120 may displace the carrier 140 along and past the carrier engaging housing lugs 118, and upon release of the actuator 120, the carrier 140 may remain engaged with the most-recently passed carrier engaging housing lug 118. Thus, increments of displacement of the carrier 140 may correspond to the spacing of the carrier engaging housing lugs 118, rather than the length of the stroke of the actuator 120.

As the actuator 120 is released following the stroke, interaction between the spring 115, the housing 110, and the actuator 120 will return the actuator 120 to the unconstrained position (the position shown in FIG. 3) as discussed above. Corresponding rotation of the actuator 120 about the pin aperture 122 will thus correlate to displacement of the ratchet slide engaging portion 124, including a component of displacement in the distal direction. Interaction between the ratchet slide engaging portion 124 and the actuator engaging opening 134 will then correlate to distal displacement of the ratchet slide 130. Thus, when the actuator 120 is released at the end of a stroke, the actuator 120, the spring 115, and the ratchet slide 130 return to the same positions relative to the housing 110 as shown in FIG. 3.

As the actuator 120 returns to the unconstrained position, however, interaction between the housing engaging arm 148 and the carrier engaging housing lug 118 prevents distal displacement of the carrier 140. Specifically, the distal surface of the angled portion 149 of the housing engaging arm 148 will be in contact with a proximal facing surface of a carrier engaging housing lug 118, the interaction preventing the carrier 140 from returning to the pre-stroke position. In the exemplary stroke discussed above, the distal most carrier engaging housing lug 118a displaced the housing engaging arm 148 during the stroke, and the housing engaging arm 148 engaged with the distal most carrier engaging housing lug 118a following the stroke. Subsequent strokes move the carrier 140 along the plurality of carrier engaging housing lugs 118 in a proximal direction.

As the actuator 120 returns to the unconstrained state, radially inward displacement of the ratchet slide engaging arm 146 of the carrier 140 allows the ratchet slide 130 to move distally with respect to the carrier 140, as engagement between the carrier 140 and the carrier engaging housing lugs 118 arrest distal displacement of the carrier 140.

Referring to FIGS. 3-7, with particular reference to the view of FIG. 6, distal displacement of the ratchet slide 130 with respect to the carrier 140 creates interaction between the carrier engaging ratchet lugs 136 and the angled portion 147 of the ratchet slide engaging arm 146 causing the ratchet slide engaging arm 146 to displace radially inward. The proximal facing surface of the carrier engaging ratchet lugs 136 may be angled to facilitate this interaction. During depression of the actuator 120, engagement between the distal most carrier engaging ratchet lug 136a can displace the carrier 140 in a proximal direction; during the return of the actuator 120, another carrier engaging ratchet lug 136 (in a proximal direction) can cause the radially inward displacement of the ratchet slide engaging arm 146 until the angled portion 147 of the ratchet slide engaging arm 146 is proximal of that carrier engaging ratchet lug 136. At that point, the ratchet slide engaging arm 146 returns to a radially outward position (analogous to that shown in FIG. 6) though the distal surface of the angled portion 147 of the ratchet slide engaging arm 146 is now engaged with a proximal face of another carrier engaging ratchet lug 136 (again in a proximal direction).

During a full stroke, engagement between a first carrier engaging ratchet lug 136 can displace the carrier 140 in a proximal direction. During the return of the actuator 120, a plurality of the next carrier engaging ratchet lugs 136 (in a proximal direction) can cause a plurality of radially inward displacements of the ratchet slide engaging arm 146 as the carrier 140 remains stationary in relation to distal movement of the ratchet slide 130 and the plurality of the carrier engaging ratchet lugs 136. After the return, the angled portion 147 of the ratchet slide engaging arm 146 returns to a radially outward position (analogous to that shown in FIG. 6) though the distal surface of the angled portion 147 of ratchet slide engaging arm 146 is now engaged with a proximal face of a second carrier engaging ratchet lug 136 (again in a proximal direction). In such a configuration, a plurality of carrier engaging ratchet lugs 136 may be disposed between the first carrier engaging ratchet lug 136 engaged during the stroke and the second carrier engaging ratchet lug 136 engaged at the end of that same stroke. For example, 1, 2, 3, 4, 5, 6, or more carrier engaging ratchet lugs 136 may be disposed between the first carrier engaging ratchet lug 136 engaged during a single stroke and the second carrier engaging ratchet lug 136 engaged at the end of that single stroke.

Displacement of the ratchet slide 130 sufficient to move to engagement with a subsequent carrier engaging ratchet lug 136 may correspond with the magnitude of displacement of the ratchet slide 130 corresponding to a return of the actuator 120. One return of the actuator 120 following at least a partial stroke can move the ratchet slide 130 such that a plurality of carrier engaging ratchet lugs 136 may serially engage the carrier 140 during the stroke.

Accordingly, as described above, depressing the actuator 120 for a full stroke, then allowing the actuator 120 to return to the unconstrained position, displaces the carrier 140 with respect to the housing 110 in discrete increments, corresponding to the distance between a plurality of carrier engaging housing lugs 118 along the longitudinal direction. Depressing the actuator 120 for a partial stroke, then allowing the actuator 120 to return to the unconstrained position, can displace the carrier 140 with respect to the housing 110 in discrete increments, corresponding to the distance between adjacent carrier engaging housing lugs 118 along the longitudinal direction.

As detailed below, the relative position of the carrier 140 with respect to the housing 110 may correlate to the degree of deployment of a stent from the deployment device 100. Thus, visual, audible, and tactile feedback as to the position of the carrier 140 provides a user with information regarding stent deployment during use of the deployment device 100. This information may correlate to increased control during deployment as the practitioner quickly and intuitively can surmise the degree of stent deployment.

In some configurations, at least the outer sheath 150 of the elongate delivery catheter assembly 104 may be displaced proximally relative to the inner sheath 160 to deploy the stent during use of the deployment device 100. The configuration of the deployment device 100 (e.g., comprising the semi-continuous disposition of the plurality of the carrier engaging ratchet lugs 136) can allow or permit more than one increment of displacement of the carrier 140 in relation to the ratchet slide 130. Furthermore, the configuration of the deployment device 100 can allow or permit finely tuned deployment of the stent. For example, the stent can be deployed in about a 1 mm increment, about a 2 mm increment, about a 3 mm increment, about a 4 mm increment, about a 5 mm increment, or any other suitable increment.

The increments of displacement of the carrier 140 may be about 0.5 mm, about 1 mm, about 2 mm, about 3 mm, about 4 mm, about 5 mm, about 10 mm, about 25 mm, about 50 mm, about 100 mm, or any other suitable increment of displacement. The incremental displacement of the carrier 140 may further facilitate partial deployment of a stent, allowing a practitioner to deploy the stent in increments, potentially adjusting or confirming the position of the stent between these increments.

FIGS. 2 and 3 illustrate the distal deployment button 170 disposed adjacent a distal end of the handle assembly 102. The distal deployment button 170 can be configured to prevent or restrict proximal movement of the carrier 140 while the stent deployment device 100 is shipped from a manufacturing facility to a healthcare facility and prior to deployment of the stent into the patient during the stent deployment procedure. The distal deployment button 170 is slidingly disposed orthogonally to the longitudinal axis of the handle assembly 102 within a distal passage 126 such that opposing ends of the distal deployment button 170 extend outside of the housing 110. In a non-actuated position, the distal deployment button 170 prevents or restricts proximal movement of the carrier 140 when the actuator 120 is depressed. In an actuated position when the distal deployment button 170 is displaced orthogonally to the longitudinal axis of the handle assembly 102, the distal deployment button 170 allows proximal movement of the carrier 140 when the actuator 120 is depressed to deploy a distal portion of the stent. In another embodiment, when the distal deployment button 170 is in the actuated position a first stent may be fully deployed.

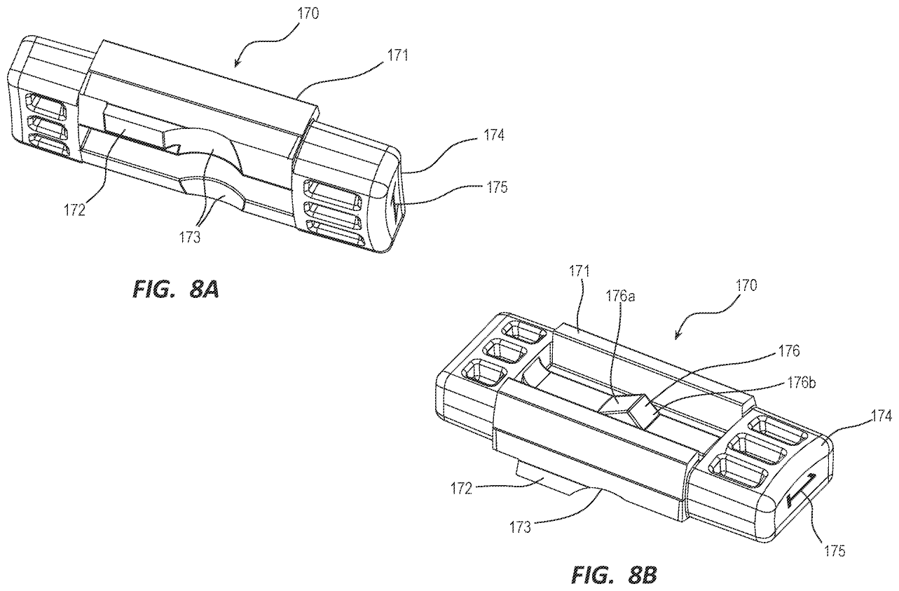

FIGS. 8A and 8B illustrate an embodiment of the distal deployment button 170. As illustrated, the distal deployment button 170 includes a body 171, a protrusion 172, a recess 173, and a button end 174. The protrusion 172 extends downward from the body 171 and is configured to engage a carrier protrusion 145 (shown in FIG. 5) of the carrier 140 to prevent or restrict proximal movement of the carrier 140 when the distal deployment button 170 is in the non-actuated position. As depicted in FIG. 5, the carrier protrusion 145 extends radially outward from the carrier 140 and has an arcuate shape that extends along the length of the carrier 140. In other embodiments, the carrier protrusion 145 may be a tab having an arcuate, square, rectangular, or any other suitable shape. The recess 173 is disposed adjacent to the protrusion 172 and has an arcuate shape configured to match the arcuate shape of the carrier protrusion 145. In another embodiment, the shape of the recess 173 may match any suitable shape of the carrier protrusion 145. When the distal deployment button 170 is in the actuated position, the carrier 140 is allowed to be moved proximally by the ratchet slide 130 as the carrier protrusion 145 aligns with and is received into the recess 173.

The body 171 includes the button end 174 configured to be engaged by a user's finger to displace the distal deployment button 170 transversely from the non-actuated position to the actuated position. The button end 174 may include an indicium 175 (e.g., number “1”) to indicate a sequence of actuations of the deployment members 170, 180. The number “1” indicium indicates that the distal deployment button 170 would be actuated prior to the proximal deployment button 180. The indicium 175 may be transfer printed onto the button end 174. In another embodiment, the indicium 175 may be an adhesive label.

A detent 176 may engage with the housing 110 to provide an increased force needed to translate the distal deployment button 170 between the non-actuated state and the actuated state. The detent 176 can prevent inadvertent actuation of the distal deployment button 170 resulting in premature deployment of the stent. As depicted in FIG. 8B, the detent 176 includes a triangular shape having opposing angled surfaces 176a, 176b. An angle of the surface 176a is greater than the angle of the surface 176b. In this configuration, the force needed to translate the distal deployment button 170 to the actuated position may be greater than the force needed to return the distal deployment button 170 to the non-actuated position. In certain embodiments, the angle of surface 176b may be about 90 degrees to prevent return of the distal deployment button 170 to the non-actuated position. In another embodiment, the detent 176 may include an arcuate shape.

FIGS. 2 and 3 illustrate the proximal deployment button 180 operably coupled to the housing 110 proximal to the distal deployment button 170. The proximal deployment button 180 can be configured to prevent or restrict proximal movement of the carrier 140 to prevent or restrict deployment of a proximal portion of the stent. In some embodiments, the proximal deployment button 180 may prevent or restrict deployment of a second, third, or fourth stent. A body 181 of the proximal deployment button 180 is slidingly disposed orthogonally to the longitudinal axis of the handle assembly 102 within a proximal passage 127 such that opposing ends of the proximal deployment button 180 extend outside of the housing 110. In a non-actuated position, the proximal deployment button 180 prevents or restricts proximal movement of the carrier 140 when the actuator 120 is depressed. In an actuated position when the body 181 is displaced orthogonally, the proximal deployment button 180 allows proximal movement of the carrier 140 when the actuator 120 is depressed to deploy a proximal portion of the stent. In another embodiment, when the proximal deployment button 180 is in the actuated position, the second, third, or fourth stent may be fully deployed.

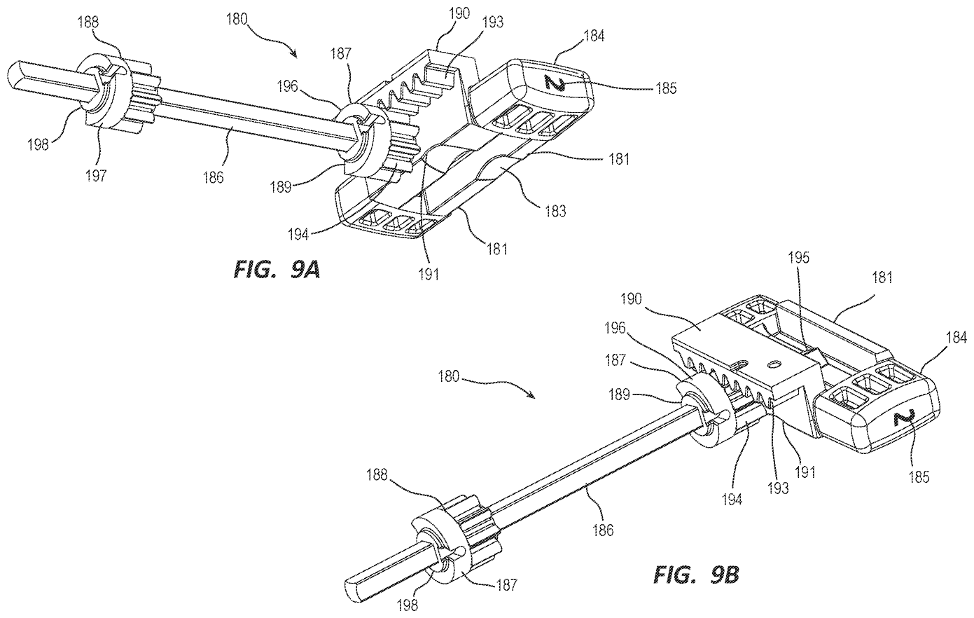

FIGS. 9A and 9B illustrate perspective views of the proximal deployment button 180. As depicted, the proximal deployment button 180 includes the body 181, a pinion gear 187, a stop member 188, and a rack gear 190. In certain embodiments, the body 181 may be substantially similar to the body 171 of the distal deployment button 170 to reduce the number of unique components of the stent deployment device 100 to reduce manufacturing costs. The body 181 includes a recess 183. The recess 183 has an arcuate shape configured to match the arcuate shape of the carrier protrusion 145 (shown in FIG. 5). In another embodiment, the shape of the recess 183 may match any suitable shape of the carrier protrusion 145. When the proximal deployment button 180 is in the actuated position, the carrier 140 is allowed to be moved proximally by the ratchet slide 130 as the carrier protrusion 145 aligns with and is received into the recess 183.

The body 181 includes a button end 184 configured to be engaged by a user's finger to displace the body 181 transversely from the non-actuated position to the actuated position. The button end 184 may include an indicium 185 (e.g., number “2”) to indicate a sequence of actuations of the deployment buttons 170, 180. The number “2” indicium indicates that the proximal deployment button 180 would be actuated following actuation of the distal deployment button 170. The indicium 185 may be transfer printed onto the button end 184. In another embodiment, the indicium 185 may be an adhesive label. The body 181 may include a detent 195 configured to be substantially similar to the detent 176 of the body 171 in form and function.

The rack gear 190 is coupled to the body 181. In some embodiments, the body 181 and the rack gear 190 may be separate components couplable together using any suitable technique, such as a snap fit, welding, bonding, gluing, etc. In other embodiments, the body 181 and rack gear 190 may be a unibody construct. The rack gear 190 includes a recess 191 disposed in a bottom surface. As shown, the recess 191 has an arcuate shape configured to match the arcuate shape of the carrier protrusion 145 (shown in FIG. 5). In another embodiment, the shape of the recess 191 may match any suitable shape of the carrier protrusion 145. When the proximal deployment button 180 is in the actuated position, the recesses 183, 191 are aligned to allow the carrier 140 to be moved proximally by the ratchet slide 130 as the carrier protrusion 145 aligns with and is received into the recesses 183, 191.

The pinion gear 187 is operably coupled to the rack gear 190. Gear teeth 193 of the rack gear 190 engage with gear teeth 194 of the pinion gear 187 to rotate the pinion gear 187 when the body 181 and rack gear 190 are displaced transversely to actuate the proximal deployment button 180. The pinion gear 187 may be rotated from about zero degrees to about 100 degrees by the rack gear 190, and may be rotated about 60 degrees to about 100 degrees, or about 90 degrees by the rack gear 190.

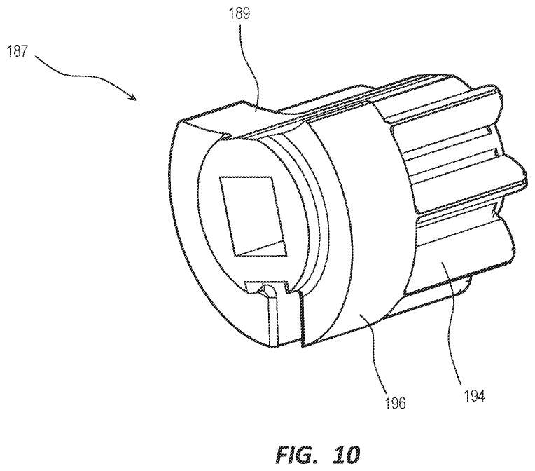

As illustrated in FIG. 10, the pinion gear 187 includes a rim 196, a notch 189 disposed in the rim 196, and the gear teeth 194. The rim 196 extends radially outward and is configured to engage with the carrier protrusion 145 to prevent or restrict proximal movement of the carrier 140 when the proximal deployment button 180 is in the non-actuated position. The notch 189 has an arcuate shape configured to match the arcuate shape of the carrier protrusion 145 (shown in FIG. 5). In another embodiment, the shape of the notch 189 may match any suitable shape of the carrier protrusion 145. When the proximal deployment button 180 is in the actuated position, the pinion gear 187 is rotated such that the notch 189 and the recesses 183, 191 are aligned to allow the carrier 140 to be moved proximally by the ratchet slide 130 as the carrier protrusion 145 aligns with and is received into one or more of the notches 189 and the recesses 183, 191.

As depicted in FIGS. 9A and 9B, an axle or rod 186 is coupled to and extends distally from the pinion gear 187. The axle 186 has a polygonal cross-sectional shape (e.g., square) to provide positive rotational coupling of the axle 186 to the pinion gear 187 and the stop member 188. The stop member 188 can be rotated by the axle 186 between about 60 degrees and 100 degrees and can be rotated about 90 degrees. The stop member 188 has a circular shape and includes a rim 197 and a notch 198 disposed in the rim 197. In some embodiments, the stop member 188 may be substantially similar to the pinion gear 187 to reduce the number of unique components of the stent deployment device 100 to reduce manufacturing costs. The rim 197 extends radially outward and is configured to engage with the carrier protrusion 145 to prevent or restrict proximal movement of the carrier 140 when the proximal deployment button 180 is in the non-actuated position. The notch 198 has an arcuate shape configured to match the arcuate shape of the carrier protrusion 145 (shown in FIG. 5). In another embodiment, the shape of the notch 198 may match any suitable shape of the carrier protrusion 145. When the proximal deployment button 180 is in the actuated position, the stop member 188 is rotated such that the notches 189, 198 and the recesses 183, 191 are aligned to allow the carrier 140 to be moved proximally by the ratchet slide 130 as the carrier protrusion 145 aligns with and is received into one or more of the notches 189, 198 and the recesses 183, 191.

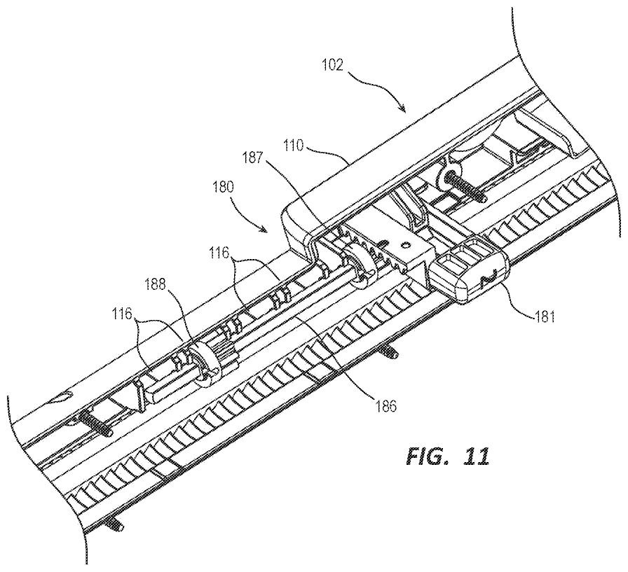

FIG. 11 is a perspective view of a portion of the handle assembly 102 with a portion of the housing 110 removed to expose internal components. As illustrated, the proximal deployment button 180 is disposed within the housing 110 with the axle 186 extending distally from the body 181 and the pinion gear 187. The housing 110 includes one or more slots 116. In the depicted embodiment, the number of slots 116 is four. In other embodiments, the number of slots 116 can be fewer or greater than four. The stop member 188 is selectively disposed within one of the slots 116 defining a distance between the distal deployment button 170 and the stop member 188 that the carrier protrusion 145 can be moved without a positive stop. The slots 116 allow rotational movement and prevent axial movement of the stop member 188. The distance may range from about 15 millimeters to about 275 millimeters. The slots 116 can be equidistantly spaced with a distance of about 8 mm to about 12 mm, or about 8 mm to about 10 mm between each of the slots 116. In some embodiments, the position of the stop member 188 within one of the slots 116 is determined at the time of manufacture of the stent deployment device 100 based on the length of the stent and the stent deployment device 100 is provided to the user in a pre-set configuration. In another embodiment, the position of the stop member 188 within one of the slots 116 may be determined at the time of use by the user.

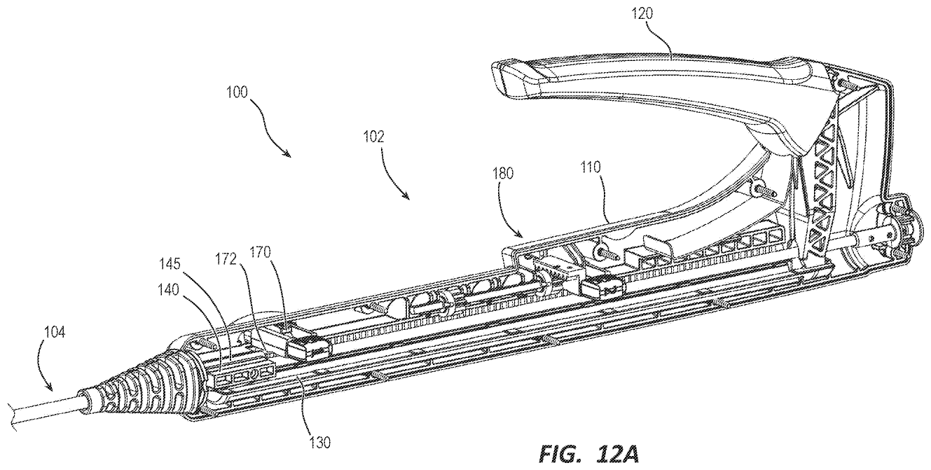

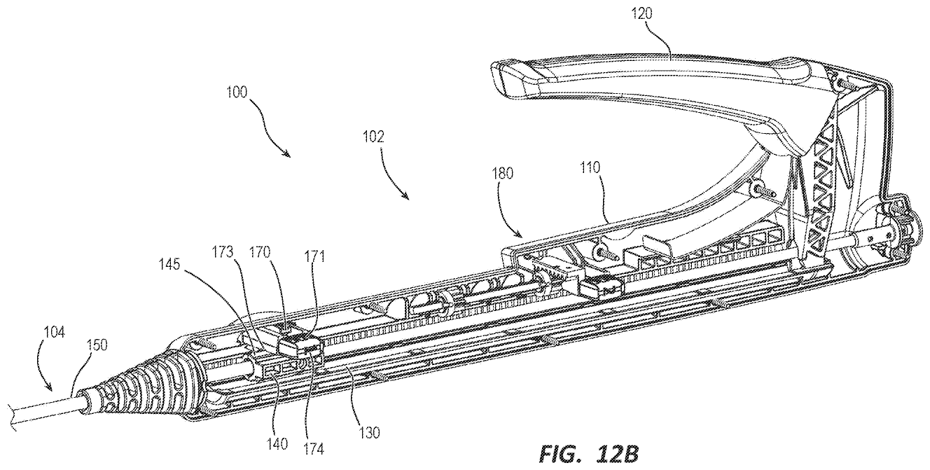

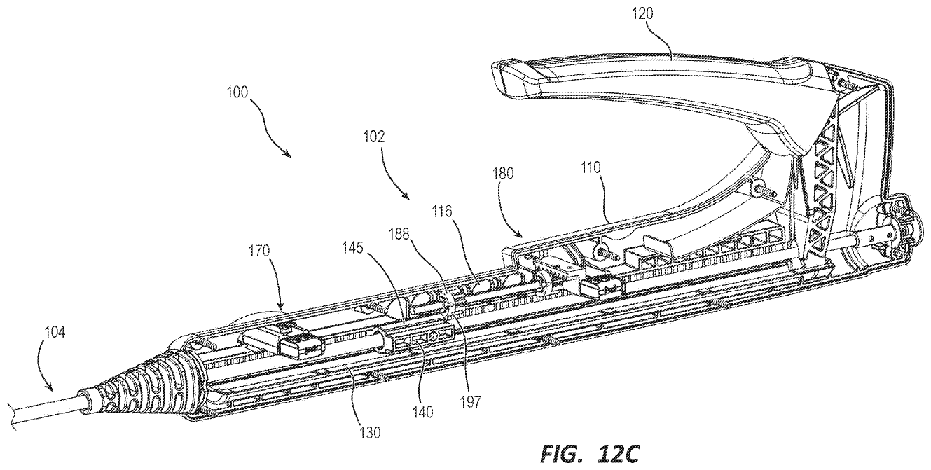

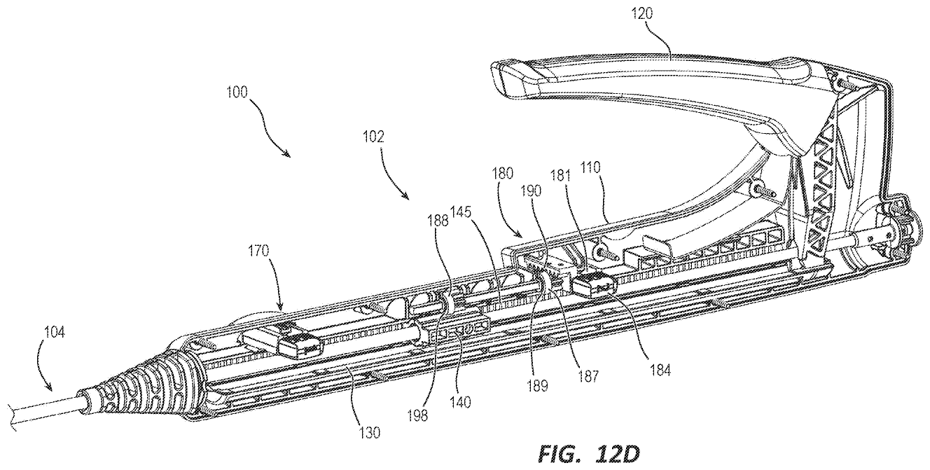

FIGS. 12A-12D illustrate a method of using the stent deployment device 100. The handle assembly 102 is shown with a portion of the housing 110 removed to display internal components. As illustrated in FIG. 12A, the stent deployment device 100 is in a stent pre-deployment state. The carrier 140 is disposed at a distal end of the housing 110 and coupled to the ratchet slide 130 as previously described. The distal deployment button 170 is in the locked or non-actuated position wherein the carrier protrusion 145 engages the protrusion 172 to prevent or restrict proximal movement of the carrier 140 by the ratchet slide 130 when the actuator 120 is depressed. In some embodiments, depression of the actuator 120 is substantially prevented or restricted in such a state. The proximal deployment button 180 is also in the locked or non-actuated state.

As illustrated in FIG. 12B, the distal deployment button 170 is in the unlocked or actuated position wherein the body 171 is transversely displaced by a user's finger in engagement with the button end 174. The recess 173 is in alignment with the carrier protrusion 145 allowing the carrier 140 to be proximally displaced by the ratchet slide 130 when the actuator 120 is depressed by the user. As the carrier 140 is displaced proximally, the outer sheath 150 is proximally displaced relative to the stent disposed at the distal end of the delivery catheter assembly 104, allowing a distal portion of the stent to be deployed. In another embodiment, as the carrier 140 is displaced proximally, a first stent of the delivery catheter assembly 104 can be deployed.

As illustrated in FIG. 12C, the proximal deployment button 180 is in the locked or non-actuated position. The stop member 188 is disposed in one of the slots 116 of the housing 110 that was determined at the time of manufacture of the stent deployment device 100 or by the user prior to the stent deployment procedure. The determination of which of the slots 116 to place the stop member 188 into may be based on a distance from the distal deployment button 170 to the stop member 188 that corresponds to approximately one half of the length of the stent or to the length of a first stent. The carrier protrusion 145 is engaged with the rim 197 of the stop member 188 to prevent proximal movement of the carrier 140 when the actuator 120 is depressed. The user may confirm appropriate deployment of the distal portion of the stent or of the first stent when the carrier 140 is prevented or restricted from proximal movement by the stop member 188.

As illustrated in FIG. 12D, the proximal deployment button 180 is in the unlocked or actuated position wherein the body 181 is transversely displaced relative to the housing 110 by a user's finger in engagement with the button end 184. The indicium 185 is transversely displaced relative to the pinion gear 187 to rotate the pinion gear 187 and the stop member 188 wherein the notches 189, 198 are in alignment with the recesses 183, 191 (shown in FIG. 9A) to allow proximal movement of the carrier 140 by the ratchet slide 130 when the actuator 120 is depressed. As the carrier 140 is displaced proximally, the proximal portion of the stent of the delivery catheter assembly 104 is deployed. In another embodiment, as the carrier 140 is displaced proximally, a second stent of the delivery catheter assembly 104 can be deployed.

FIGS. 13A and 13B depict an embodiment of a stent deployment device 200 that resembles the stent deployment device 100 described above in certain respects. Accordingly, like features are designated with like reference numerals, with the leading digit incremented to “2.” For example, the embodiment depicted in FIGS. 13A and 13B includes a proximal deployment member 280 that may, in some respects, resemble the proximal deployment button 180 of FIG. 3. Relevant disclosure set forth above regarding similarly identified features thus may not be repeated hereafter. Moreover, specific features of the stent deployment device 100 and related components shown in FIGS. 1-12D may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the stent deployment device 200 and related components depicted in FIGS. 13A and 13B. Any suitable combination of the features, and variations of the same, described with respect to the stent deployment device 100 and related components illustrated in FIGS. 1-12D can be employed with the stent deployment device 200 and related components of FIGS. 13A and 13B, and vice versa. This pattern of disclosure applies equally to further embodiments depicted in subsequent figures and described hereafter, wherein the leading digits may be further incremented.

As illustrated in FIG. 13A, a proximal deployment member 280 of an embodiment of a stent deployment device 200 is in the locked or non-actuated position. A stop member of the proximal deployment member 280 has been removed to allow a carrier 240 to be moved proximally between a distal deployment member 270 and a pinion gear 287 of the proximal deployment member 280. A distance from the distal deployment member 270 to the pinion gear 287 may correspond to approximately one half of the length of the stent or to the length of a first stent. The carrier protrusion 245 is engaged with the rim 296 of the pinion gear 287 to prevent proximal movement of the carrier 240 when the actuator 220 is depressed. The user may confirm appropriate deployment of the distal portion of the stent or of the first stent when the carrier 240 is prevented or restricted from proximal movement by the pinion gear 287.

As illustrated in FIG. 13B, the proximal deployment member 280 is in the unlocked or actuated position wherein the body 281 is transversely displaced relative to the housing 210 by a user's finger in engagement with the button end 284. The rack gear 290 is transversely displaced relative to the pinion gear 287 to rotate the pinion gear 287 wherein the notch 289 is in alignment with the recesses 283, 291 (shown in FIGS. 9A and 9B) to allow proximal movement of the carrier 240 by the ratchet slide 230 when the actuator 220 is depressed. As the carrier 240 is displaced proximally, the proximal portion of the stent of the delivery catheter assembly 204 can be deployed. In another embodiment, as the carrier 240 is displaced proximally, a second stent of the delivery catheter assembly 204 can be deployed.

As illustrated in FIG. 14A, a proximal deployment member 380 of an embodiment of a stent deployment device 300 is in the locked or non-actuated position. A stop member, pinion gear, and rack gear of the proximal deployment member 380 have been removed to allow a carrier 340 to be moved proximally between a distal deployment button 370 and a body 381 of the proximal deployment member 380. A distance from the distal deployment button 370 to the body 381 may correspond to approximately one half of the length of the stent or to the length of a first stent. The carrier protrusion 345 is engaged with a protrusion 372 to prevent proximal movement of the carrier 340 when the actuator 320 is depressed. The user may confirm appropriate deployment of the distal portion of the stent or of the first stent when the carrier 340 is prevented or restricted from proximal movement by the body 381.

As illustrated in FIG. 14B, the proximal deployment button 380 is in the unlocked or actuated position wherein the body 381 is transversely displaced relative to the housing 310 by a user's finger in engagement with the button end 384. A recess 383 is aligned with the carrier protrusion 345 to allow proximal movement of the carrier 340 by the ratchet slide 330 when the actuator 320 is depressed. As the carrier 340 is displaced proximally, the proximal portion of the stent of the delivery catheter assembly 304 is deployed. In another embodiment, as the carrier 340 is displaced proximally, a second stent of the delivery catheter assembly 304 can be deployed.

In certain embodiments, the distance between the distal deployment button 370 and the proximal deployment button 380 may be approximately equivalent to one half of the length of the stent or to the length of a first stent, such that a position of the deployed distal portion of the stent or the deployed first stent may be confirmed by the user prior to actuation of the proximal deployment button 380 and deployment of the proximal portion of the stent or a second stent.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of deploying a stent may include one or more of the following steps: disposing a stop member of a second deployment member within a deployment member slot; actuating a first deployment member to allow proximal movement of a carrier coupled to an outer sheath of a catheter deployment assembly; actuating an actuator to proximally incrementally displace the carrier and the outer sheath to deploy a distal portion of the stent; engaging the carrier with the second deployment member to prevent proximal movement of the carrier and the outer sheath to prevent deployment of a proximal portion of the stent; and actuating the second deployment member to allow proximal movement of the carrier and the outer sheath to deploy the proximal portion of the stent. Other steps are also contemplated.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a stent deployment device of this disclosure, the proximal end of the stent deployment device refers to the end nearest to the handle assembly and the distal end refers to the opposite end, the end nearest to the stent.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially equivalent” is recited with respect to a feature, it is understood that in further embodiments, the feature can have a precisely equivalent configuration.

The terms “a” and “an” can be described as one, but not limited to one. For example, although the disclosure may recite a housing having “a carrier,” the disclosure also contemplates that the housing can have two or more carriers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A prosthesis deployment device comprising:
an elongate delivery catheter assembly configured to retain and deploy a prosthesis;
a housing assembly operably coupled to the delivery catheter assembly, wherein the housing assembly comprises:
a housing coupled to the delivery catheter assembly;
an actuator;
a slide configured to be moved proximally by the actuator when the actuator is depressed and pivots on portions of the housing assembly;
a carrier coupled to a portion of the delivery catheter assembly and configured to be moved proximally by the slide, wherein the carrier is configured to proximally displace the portion of the delivery catheter to deploy a prosthesis;
a first deployment button configured to engage the carrier to prevent deployment of a distal portion of the prosthesis; and
a second deployment button configured to engage the carrier to prevent deployment of a proximal portion of the prosthesis,
wherein a portion of the second safety button is disposed within one of a plurality of deployment button slots of the housing to provide a variable distance between the first deployment button and the second deployment button.

2. The device of claim 1, wherein the first deployment button comprises:
a body comprising:
a protrusion configured to engage the carrier to prevent proximal movement of the carrier; and
a recess configured to allow proximal movement of the carrier.

3. The device of claim 1, wherein the second deployment button comprises:
a body portion;
a linear actuator coupled to the body portion;
a rod coupled to and rotated by the linear actuator; and
a stop member coupled to the rod and configured to selectively prevent proximal movement of the carrier.

4. The device of claim 3, wherein the linear actuator comprises:
a linear gear coupled to the body portion; and
a pinion gear operably coupled to the linear gear,
wherein the rod is coupled to and rotated by the pinion gear.

5. The device of claim 3, wherein the stop member comprises:
a protrusion configured to engage the carrier to prevent proximal movement of the carrier; and
a recess configured to allow proximal movement of the carrier.

6. The device of claim 3, wherein the stop member is rotatable from zero degrees to 100 degrees by the linear actuator.

7. The device of claim 3, wherein the stop member is disposable within one of the plurality of deployment button slots.

8. The device of claim 1, wherein the plurality of deployment button slots comprise from zero to four slots.

9. The device of claim 1, wherein the variable distance between the first deployment button and the second deployment button ranges from 15 millimeters to 275 millimeters.

10. The device of claim 1, wherein the carrier is coupled to an outer sheath of the delivery catheter assembly.

11. The device of claim 1, wherein the actuator is configured to proximally displace the carrier in increments ranging from one millimeter to 20 millimeters.

12. A stent deliver device, comprising:
a handle assembly comprising:
a housing comprising:
a plurality of carrier engaging housing lugs; and
a plurality of deployment member slots;
an actuator operably coupled to the housing comprising:
an input portion; and
a transfer arm comprising a ratchet slide engaging portion;
a spring coupled to the housing and the actuator;
a rachet slide slidingly disposed within the housing comprising:
a plurality of carrier engaging ratchet lugs; and
an actuator engaging opening configured to couple with the ratchet slide engaging portion;
a carrier comprising:
a ratchet slide engaging arm configured to engage with the plurality of carrier engaging ratchet lugs to provide incremental proximal movement of the carrier; and
a housing engaging arm configured to engage with the plurality of carrier engaging housing lugs to prevent distal movement of the carrier;
a first safety member configured to selectively restrain proximal movement of the carrier at a distal position; and
a second safety member configured to selectively restrain proximal movement of the carrier at a proximal position,
wherein the second safety member is configured to be selectively positioned in one of the plurality of deployment member slots.

13. The device of claim 12, further comprising:
a delivery catheter assembly extending distally from the handle assembly, comprising:
an outer sheath coupled to the carrier;
an inner sheath disposed within the outer sheath and coupled to the housing; and
one or more stents disposed between the outer sheath and the inner sheath at a distal end of the inner sheath.

14. The device of claim 13,
wherein when the carrier is in the distal position, deployment of a distal portion of the stent is prevented, and
wherein when the carrier is in the proximal position, deployment of a proximal portion of the stent is prevented.

15. The device of claim 12, wherein the first safety member comprises:
a body comprising:
a protrusion configured to engage the carrier to prevent proximal movement of the carrier; and
a recess configured to allow proximal movement of the carrier.

16. The device of claim 12, wherein the second safety member comprises:
a body portion;
a linear actuator coupled to the body portion;
a rod coupled to and rotated by the linear actuator; and
a stop member coupled to the rod and configured to selectively prevent proximal movement of the carrier.

17. The device of claim 16, wherein the stop member comprises:
a protrusion configured to engage the carrier to prevent proximal movement of the carrier; and
a recess configured to allow proximal movement of the carrier.

18. The device of claim 16, wherein the stop member is disposable within one of the plurality of deployment member slots.

19. A method of deploying a stent, comprising:
disposing a stop member of a second safety member within a deployment member slot;
actuating a first safety member to allow proximal movement of a carrier coupled to an outer sheath of a catheter deployment assembly;
actuating an actuator to proximally incrementally displace the carrier and the outer sheath to deploy a distal portion of the stent;
engaging the carrier with the second safety member to prevent proximal movement of the carrier and the outer sheath to prevent deployment of a proximal portion of the stent; and
actuating the second safety member to allow proximal movement of the carrier and the outer sheath to deploy the proximal portion of the stent.

20. The method of claim 19, wherein actuating the second safety member comprises rotating the stop member between 60 degrees to 100 degrees.

21. The method of claim 19, wherein actuating the actuator to proximally incrementally displace the carrier and the outer sheath comprises deploying a first stent.

22. The method of claim 19, wherein actuating the second safety member to allow proximal movement of the carrier and the outer sheath comprises deploying a second stent.
